(19) United States
(12) Patent Application Publication
(10) Pub. No.: US 2025/0241774 A1
(43) Pub. Date: Jul. 31, 2025

(54) HUNGER SATIATING DENTAL APPLIANCE

(71) Applicant: Jeffrey GOLDBERG, Amherst, NY (US)

(72) Inventor: Jeffrey GOLDBERG, Amherst, NY (US)

(21) Appl. No.: 18/855,028

(22) PCT Filed: Apr. 5, 2023
(86) PCT No.: PCT/US2023/065380

§ 371 Date: Oct. 8, 2024

Related U.S. Application Data
(60) Provisional application No. 63/331,721, filed on Apr. 15, 2022.

(51) Int. Cl. A61F 5/00

(52) U.S. Cl. CPC A61F 5/0006

(57) ABSTRACT

An oral appliance includes a body removably securable to one or more teeth, a chamber disposed on the body, and an active medium disposed in the chamber. The active medium produces an aroma delivered retronasally. When the oral appliance is installed in the mouth of an individual, the aroma satiates the hunger of the individual.

### CROSS-REFERENCE TO RELATED APPLICATIONS

This disclosure claims priority to U.S. Provisional Application No. 63/331,721, filed Apr. 15, 2022, the entire disclosure of which is hereby incorporated by reference herein.

### FIELD OF THE DISCLOSURE

This disclosure relates to oral appliances and, more particularly, to oral appliances worn while eating.

### BACKGROUND OF THE DISCLOSURE

Obesity is widely recognized as one of the biggest public health concerns in the world today. Approximately 34% of adults and 20% of children and adolescents in the United States are obese. Obesity affects every segment of the population, increases risks associated with many chronic diseases, and has a role virtually every imaginable aspect of health and lifestyle. The weight loss market in the United States is valued at approximately $72 billion and growing. Despite the size and diversity of this market, there remains opportunity for new innovations.

Dentists are responsible for the prescription and delivery of oral appliances or orthotics within the medical field. The wide range of established applications includes dentures and partial dentures, orthodontic appliances, mouth guards for jaw pain (temporomandibular joint disorder or TMJ) and headaches, sleep apnea appliances, athletic protective mouth guards, whitening trays, and many more. However, diet and weight management have not previously been included among the conditions addressed with oral appliances, or by dentists in general.

Therefore, what is needed is an oral appliance that can be used to assist diet and weight management treatments.

### BRIEF SUMMARY OF THE DISCLOSURE

An embodiment of the present disclosure provides an oral appliance comprising a body removably securable to one or more teeth, a chamber disposed on the body, and an active medium disposed in the chamber. The active medium may produce or deliver an aroma delivered retro-nasally.

According to an embodiment of the preset disclosure, the body may be removably securable to one or more upper teeth. The body may be removably securable to an anterior tooth and a posterior tooth. The body may be removably securable to a left-side tooth and a right-side tooth on opposite sides of the mouth. The body may include a cross member which extends across the mouth from the left-side tooth to the right-side tooth. The one or more teeth may be natural teeth or part of a denture or partial denture. The body may be manufactured based on a custom mold of the one or more teeth. The body may include a clasp which surrounds sides of the one or more teeth, such that occlusal surfaces of the one or more teeth are uncovered.

According to an embodiment of the present disclosure, the chamber may be fixedly secured to the body. The chamber may be removably secured to the body. The chamber may be integrally formed with the body. The chamber may be positioned parallel to the one or more teeth or angled relative to the one or more teeth. The chamber may have a rounded surface. The chamber may comprise a sealable opening which, when open, is configured to receive the active medium, and when closed, is configured to retain the active medium within the chamber. The chamber may comprises a porous surface, and the aroma produced by the active medium may exit the chamber via the porous surface. The chamber may comprise a first chamber and a second chamber separate from the first chamber. The first chamber and the second chamber may be disposed on opposite sides of the mouth or on the same side of the mouth.

According to an embodiment of the present disclosure, the active medium may produce or release the aroma by interaction with an activator. The activator may be saliva or a component of saliva. The activator may be an initiating solution. The aroma may be a hunger-satiating aroma. The active medium may be a solid, gel, paste, or liquid. The active medium may produce or release one or more of the following aromas: chocolate, vanilla, cinnamon, floral, butter, lemon, menthol, or mint. Without intending to be bound by any particular theory, these aromas may be considered to satiate hunger.

An embodiment of the present disclosure provides a method of satiating hunger in an individual comprising: installing an oral appliance in the mouth of the individual, and engaging the active medium to produce or release the aroma that is detected retro-nasally by the individual. The aroma may satiate the hunger of the individual when detected retro-nasally.

### DESCRIPTION OF THE DRAWINGS

For a fuller understanding of the nature and objects of the disclosure, reference should be made to the following detailed description taken in conjunction with the accompanying drawings, in which:

FIG. 1 is a diagram of a human upper-respiratory system containing an oral appliance according to an embodiment of the present disclosure;

FIG. 2 illustrates a maxillary view of an oral appliance according to an embodiment of the present disclosure:

FIG. 3A illustrates a maxillary view of a body of the oral appliance of FIG. 2;

FIG. 3B illustrates a maxillary view of a body of an oral appliance according to another embodiment of the present disclosure:

FIG. 4 illustrates a maxillary view of an oral appliance according to another embodiment of the present disclosure;

FIG. 5 illustrates a lingual side view of a chamber of an oral appliance according to an embodiment of the present disclosure;

FIG. 6 illustrates a lingual side view of a chamber of an oral appliance according to another embodiment of the present disclosure; and

FIG. 7 is a flow chart of a method according to an embodiment of the present disclosure.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure. Accordingly, the scope of the disclosure is defined only by reference to the appended claims.

As used herein, oral care specialist may refer to dentists, orthodontists, dental hygienists, oral surgeon, or any other dental or oral technicians or staff members.

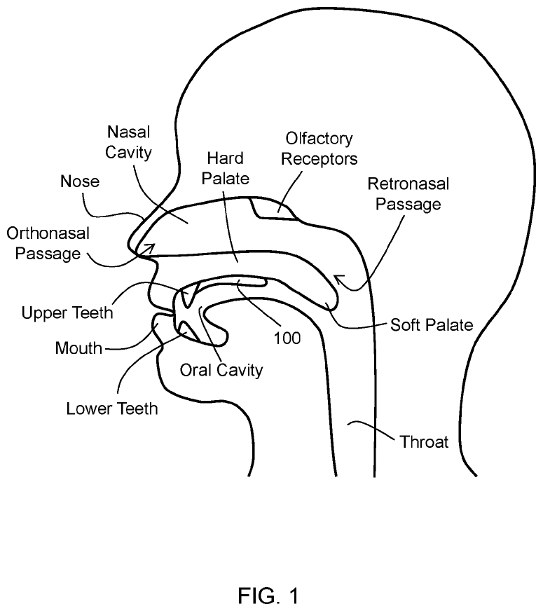

An embodiment of the present disclosure provides an oral appliance 100. The oral appliance 100 may be worn in the oral cavity of a patient or an individual. For example, FIG. 1 illustrates major structures of the patient's upper respiratory tract, which includes a nasal cavity, an oral cavity, and a throat. The oral cavity connects the mouth to the throat, and the nasal cavity connects the nose to the throat. The nasal cavity includes an orthonasal passage at the nose, and a retronasal passage at the throat. The retronasal passage is proximal to the connection between the oral cavity and the throat. The nasal cavity includes olfactory receptors which sense aromas delivered through the orthonasal passage and the retronasal passage. The oral cavity and the nasal cavity are separated by a hard palate proximal to the mouth, and a soft palate proximal to the throat. The oral appliance 100 may rest on the hard palate. The oral appliance 100 may be as far back in the oral cavity as possible, so as to be proximal to the retronasal passage.

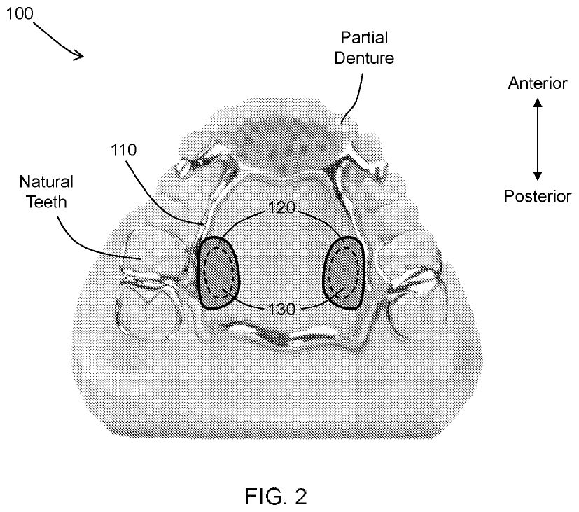

As shown in FIG. 2, the oral appliance 100 may comprise a body 110, a chamber 120 disposed on the body 110, and an active medium 130 disposed in the chamber. With the oral appliance 100 of the present disclosure, the active medium 130 may produce or release an aroma that is delivered to or detected retro-nasally by an individual. In other words, the aroma may travel through the retronasal passage to the nasal cavity, where the olfactory receptors of the individual sense the aroma.

The body 110 may be designed or based on an impression of the upper and lower teeth of the patient or individual. The impression may be produced using traditional molding techniques or digital imaging techniques. Impressions of the upper and lower teeth may allow evaluation of the patient's bite, which can ensure that the body 110 does not interfere with the bite. Use of the impression to design the body 110 may allow the oral appliance 100 to be custom fit to the anatomy of the patient, for improved comfort and function. The oral appliance 100 may not alter the alignment of the teeth. In some embodiments, the oral appliance 100 may alter or correct teeth alignment.

The body 110 may be removably securable to one or more teeth. For example, the body 110 may be removably securable to one or more upper teeth. While the body 110 may also be removably securable to one or more lower teeth, proximity of the body 110 to the retronasal passage when removably secured to one or more upper teeth may be advantageous compared to when removably secured to one or more lower teeth. The body 110 may be removably securable to the one or more teeth by a dentist, orthodontist, or any other oral care specialist. For example, the dentist, orthodontist, or other oral care specialist may install the oral appliance 100 in the patient's oral cavity, and the oral appliance 100 may remain in the patient's oral cavity until the dentist, orthodontist, or other oral care specialist removes the oral appliance 100. In this way, the oral appliance 100 may be installed similarly to semi-permanent dental devices, such as braces. Alternatively, the body 110 may be removably securable to the one or more teeth by the patient. For example, the patient may install and remove the oral appliance 100 from their oral cavity periodically, such as while sleeping, eating, etc., at the direction of an oral care specialist. In this way, the oral appliance 100 may be installed similarly to temporary dental devices, such as retainers or mouth guards.

The body 110 may be removably securable to a plurality of teeth in the patient's oral cavity. The plurality of teeth may include two or more adjacent teeth. In other words, the body 110 may be removably securable to two adjacent teeth and/or between two adjacent teeth. The plurality of teeth may include two or more teeth on opposite sides of the mouth. In other words, the body 110 may be removably securable to teeth on the left and right sides of the patient's oral cavity. While the plurality of teeth are described as the patient's natural teeth, the plurality of teeth may include one or more teeth that are part of a denture or partial denture, or a combination of natural and artificial teeth.

In an embodiment, the body 110 may be removably secured to at least four upper teeth. For example, the body 110 may be removably securable to a left anterior tooth, a right anterior tooth, a left posterior tooth, and a right posterior tooth. The at least four upper teeth may refer to areas between four pairs of upper teeth. The four upper teeth or pairs of upper teeth may be custom to the patient, as deemed suitable by a dentist, orthodontist, or other oral care specialist. With the body 110 being removably securable to the at least four upper teeth, the oral appliance 100 may be stably set and balanced within the oral cavity. The body 110 may be symmetrical (i.e., secured to the same teeth on the left and right sides of the mouth) or asymmetrical (i.e., secured to different teeth on the left and right sides of the mouth), depending on the patient's dental structure and/or oral cavity anatomy.

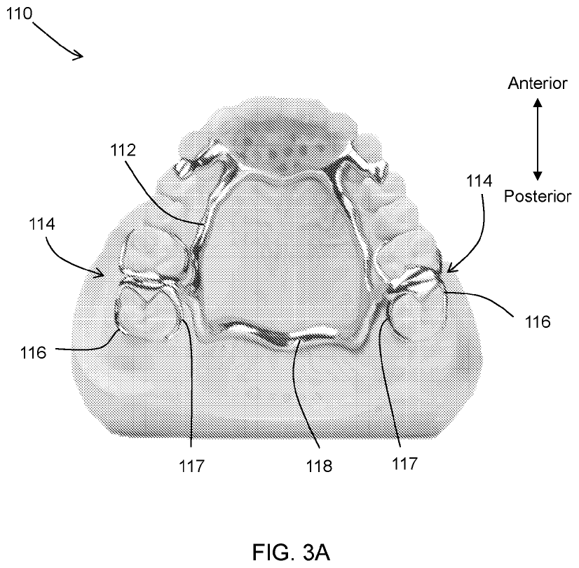

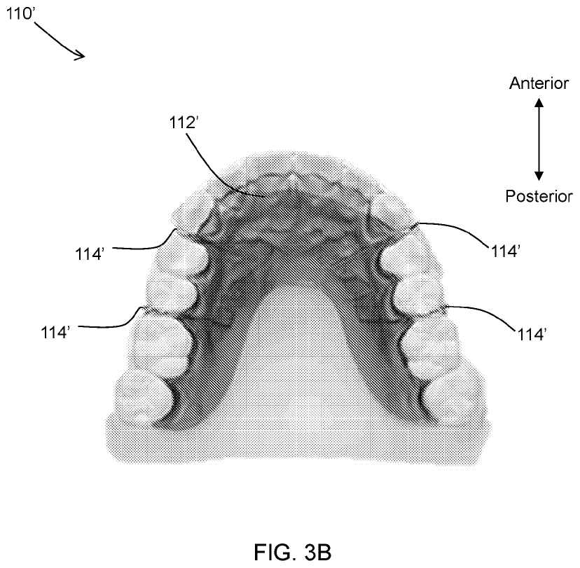

As best seen in FIG. 3A, the body 110 may include a U-shaped metal framework 112 that rests on the hard palate and clasps 114 which engage the plurality of teeth. The metal framework 112 may be U-shaped with respect to the shape of the hard palate and/or the lingual sides of the plurality of teeth. For example, the body 110 may span a portion of the dental arch or may span the entire dental arch. The clasps 114 may surround sides of the plurality of teeth, such that occlusal (i.e., biting) surfaces of the teeth are uncovered. For example, the clasps 114 may include an outer part 116 that rests against the buccal side of the tooth, apical to the crest of curvature, and an inner part 117 on the lingual side of the tooth to prevent tooth movement. The outer part 116 and the inner part 117 may be connected through the interproximal embrasure of adjacent teeth so as to not interfere with the occlusal surface of the teeth. In this way, the oral appliance 100 may be worn by the patient while eating. The inner part 117 may be connected to the metal framework 112 or may be integrally formed with the metal framework 112. The clasps 114 may be secured to some of the plurality of teeth. The clasps 114 may be wire or cast metal.

The body 110 may further include a cross member 118. The cross member 118 may extend across the hard palate. For example, the cross member 118 may extend between left and right posterior teeth. The cross member 118 may be straight or curved to the shape of the hard palate. The cross member 118 may be connected to the metal framework 112 or may be integrally formed with the metal framework 112. The cross member 118 may provide support for the body 110.

The body 110 may be manufactured using traditional casting processes.

Alternatively, computer-aided manufacturing processes can be used, such as 3D laser sintering, 3D printing, stereolithography, or milling. The body 110 may be made of a biocompatible metal or other materials. For example, the body 110 may be made of one or more materials such as metal alloys (e.g., cobalt chromium, nickel titanium), acrylics (e.g., polymethyl methacrylate), polycarbonate, polyurethane, polyethylene, polyoxymethylene polyamide, nylon, stainless steel, vinyl, composites, or combinations thereof.

Another embodiment of the body 110′ is shown in FIG. 3B. The body 110′ may include a U-shaped acrylic framework 112′ that rests on the hard palate and clasps 114′ which engage the plurality of teeth. The body 110′ may be similar to a Hawley-style orthodontic retainer of the related art. The acrylic framework 112′ may be U-shaped with respect to the shape of the hard palate and/or the lingual sides of the plurality of teeth. For example, the body 110′ may span a portion of the dental arch or may span the entire dental arch. The clasps 114″ may be wires which extend from the acrylic framework 112′ on the lingual side of the teeth through the interproximal embrasure of adjacent teeth, and wrap upward to rest against the buccal side of the tooth. In this way, the clasps 114′ will not interfere with the occlusal surface of the teeth. Ends of the clasps 114′ may be rounded or curved to improve comfort when worn by the patient. The clasps 114′ may be at least partially embedded into the acrylic framework 112′. The clasps 114′ may be secured to some of the plurality of teeth.

Referring back to FIG. 2, the chamber 120 may be fixedly secured to the body 110. For example, the chamber 120 may be a component formed separately from the body 110. The chamber 120 may be secured to the body 110 by mechanical fastening or adhesive bonding. In this way, the chamber 120 fixed to the body 110, and may remain attached while the patient's mouth is moving (e.g., eating swallowing, talking, etc.) Alternatively, the chamber 120 may be integrally formed with the body 110. The chamber 120 may be manufactured using traditional casting processes. Alternatively, computer-aided manufacturing processes can be used, such as 3D laser sintering, 3D printing, stereo lithography, or milling.

The chamber 120 may be removably secured to the body 110. For example, the chamber 120 may be installed on the body 110 such that it may be removed by the patient, or oral care specialist. This may allow installation of chambers 120 having different sizes or shapes or configured to received different active media 130 to be installed on the same body 130. The chamber 120 may be removed from the body 110 by disengaging mechanical fasteners (e.g., clips, clasps, screws, etc.) or adhesives used to secure the chamber 120 to the body 110. In the case of mechanical fasteners, dental tools may be needed to remove the chamber 120 from the body 110 and/or to install the chamber 120 to the body 110. In the case of adhesives, an adhesive between the chamber 120 and the body 110 may be dissolved or heated to weaken the bond such that the chamber 120 is removable from the body 110, and the adhesive may be re-applied when the chamber 120 is secured to the body. Adhesives may include dental or orthodontic resins used in the art for fabrication of dentures and/or orthodontic appliances. Biomimetic adhesives may be used, for example, such as those in orthodontics to bond brackets onto teeth comprising L-3,4-dihydroxyphenylalanine (DOPA). In either case of mechanical fasteners or adhesives, the chamber 120 may be secured such that there is a low risk of detachment while the patient's mouth is moving.

The chamber 120 may be made of the same material as the body 110 or a different material. For example, the chamber 120 may be made of one or more materials such as metal alloys (e.g., cobalt chromium, nickel titanium, and the like), acrylics (e.g., polymethyl methacrylate, and the like), polycarbonate, polyurethane, polyethylene, polyoxymethylene polyamide, nylon, stainless steel, vinyl, composites, or combinations thereof.

The chamber 120 may be positioned on the lingual side of the teeth, resting against the hard palate. As shown in FIG. 5, the chamber 120 may run parallel to the teeth. Alternatively, the chamber 120 may be angled relative to the teeth, such that it is angled upward/downward and/or toward the center of the mouth. The chamber 120 may be as far posterior as possible, so as to be as close to the throat/retronasal passage as possible. For example, the chamber 120 may be positioned as far posterior as (or beyond) the distal molars. In this way, aroma may be directed toward the retronasal passage for increased sensation. The chamber 120 may be positioned such that it may be minimally visible through the patient's mouth.

The shape of the chamber 120 may be custom to the patient's unique dental structure. For example, impressions of the patient's teeth may constrain the size and shape of the chamber 120. The shape of the chamber 120 may also depend on the shape and/or type of active medium 130. In an embodiment, the chamber 120 may have a rounded surface. The rounded surface may improve comfort when the oral appliance 100 is worn by the patient, such that the oral appliance 100 may be worn for extended periods of time. For example, the shape of the chamber 120 may be a rounded rectangle, ellipsoid, ovoid, or other shapes. The shape of the chamber 120 may also be configured to direct the aroma toward the retronasal passage for more effective sensation. For example, the chamber 120 may have a funnel-like shape.

As seen in FIG. 5, the chamber 120 may comprise a sealable opening 122. When the sealable opening 122 is open, the chamber 120 may configured to receive the active medium 130. When the sealable opening 122 is closed, the chamber 120 may be configured to retain the active medium 130. For example, the chamber 120 may have a hinged flap which controls the opening and closing of the sealable opening 122. In this way, the chamber 120 may protect the active medium 130 stored within. The chamber 120 may also have a clamshell shape. The sealable opening 122 may be opened and closed using a key or other tools. Alternatively, the sealable opening 122 may be opened and closed without any key or tools (e.g., by the patient or dentist's fingers). In an embodiment, the chamber 120 may be detached from the body 110 in order to be opened. In this way, risk of accidental opening of the sealable opening 122 may be reduced.

The chamber 120 may comprise a porous surface 124. The aroma produced or released by the active medium 130 may exit the chamber 120 via the porous surface 124. The porous surface 124 may cover any surfaces of the chamber 120 that are exposed inside the oral cavity. In other words, any surfaces of the chamber 120 that is not exposed inside the oral cavity (e.g., surfaces that are connected to the body 110) may be solid/nonporous. Alternatively, all surfaces of the chamber 120 may be porous or only some surfaces of the chamber 120 may be porous. The porous surface 124 may be a mesh-like metal. The size and density of the porosity of the porous surface 124 may depend on the type of active medium 130 and/or a desired rate of release of the aroma. In this way, the chamber 120 may control the release of the aroma.

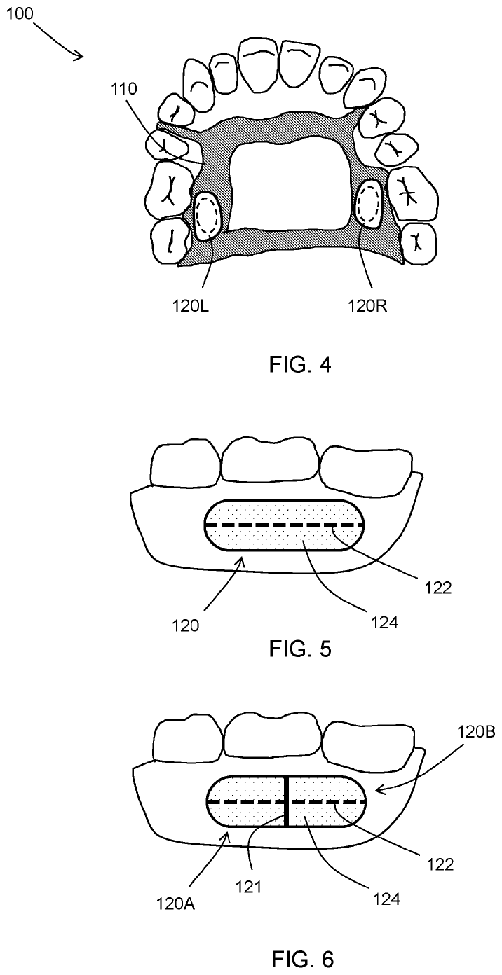

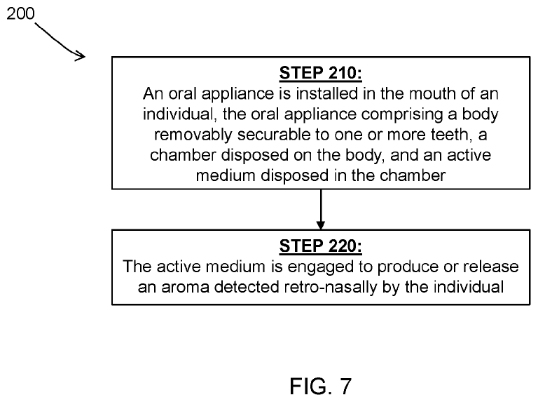

The chamber 120 may comprise a first chamber and a second chamber. The first chamber and the second chamber may be disposed on opposite sides of the mouth. For example, as shown in FIG. 4, there may be a left chamber 120L and a right chamber 120R on the left and right sides of the mouth, respectively. This may allow better distribution of the active medium 130 within the oral cavity.

In an embodiment, the first and second chamber may be on the same side of the mouth. For example, as shown in FIG. 6, the chamber 120 may comprise a first chamber 120A and a second chamber 120B. The first chamber 120A and the second chamber 120B may be separated by a barrier 121. This may allow combinations of active mediums 130 to be used in different chambers. For example, the first chamber 120A and the second chamber 120B may contain different active mediums 130; the first chamber 120A and the second chamber 120B may contain the same active medium 130; one of the first chamber 120A or the second chamber 120B may contain a combination of active mediums 130 while the other of the first chamber 120A and the second chamber 120B contains a single active medium 130; or both the first chamber 120A and the second chamber 120B each contain the same or different combinations of active medium 130. The first chamber 120A and the second chamber 120B may be the same size or different sizes. The first chamber 120A and the second chamber 120B may be arranged side-by-side (shown in FIG. 6) or on top of each other (not shown). Other arrangements of the first chamber 120A and the second chamber 120B are possible.

The active medium 130 may produce or release an aroma by interaction with an activator. The activator may enter the chamber 120 via the porous surface 124 to interact with the active medium 130. For example, the active medium 130 may produce the aroma by interaction with saliva or a component of saliva. Salivary flow and salivary composition in the patient's mouth may be tested in order to select a particular active medium 130 and volume of the active medium 130 to be disposed in the chamber 120 to produce or release an aroma having a desired effect on the patient. The activator may be an initiating solution. In other words, the active medium 130 may not produce or release an aroma under natural conditions in the oral cavity of the patient, but may produce or release an aroma upon interaction with the initiating solution. This may allow the patient to control the timing of producing or releasing the aroma in order to produce the desired effects. For example, the initiating solution may be ingested or contacted with the medium prior to eating, such that the aroma is produced/released while the patient is eating. The initiating solution may be ingested at other times, for example, when the patient is hungry.

The active medium 130 may be a solid, gel, paste, or liquid. The active medium 130 may produce or release aromas such as chocolate, vanilla, cinnamon, floral, butter, lemon, menthol, mint, or other spices. The active medium 130 may be a natural or artificial substance.

When the aroma is received or detected retro-nasally, it may yield various effects. For example, the aroma may be an appetite-suppressing aroma, which may deter the patient from eating, or a hunger-satiating aroma, which may cause the patient to feel “full” sooner. The aroma may be a flavor-enhancing aroma, which may improve the eating experience. These effects may alter the patient's diet and may contribute to a weight management treatment or plan. Other types of aromas and effects produced therefrom are within the scope of the present disclosure. For example, the aroma may deter the patient from other behaviors, such as smoking, drinking alcohol, or using recreational drugs or illicit substances. In such examples, the active medium 130 may produce or release aromas such as tobacco, alcohol, spirits, or other scents that may deter the patient from smoking or consuming alcohol or other drugs. Similarly, the aromas may simulate the consumption of alcohol (i.e. cause to taste like) when the patient is consuming non-alcoholic beverages, so as to reduce consumption of alcoholic beverages.

An embodiment of the present disclosure provides a method 200 of satiating hunger in an individual or patient. The method 200 may comprise the following steps.

At step 210, an oral appliance is installed in the mouth of the individual. The oral appliance may be the oral appliance 100 described above, comprising a body 110, chamber 120, and active medium 130, the structure of which is not repeated here. The oral appliance may be installed on a semi-permanent basis by a dentist, orthodontist, or other oral care specialist, or may be installed temporarily by the individual.

At step 220, the active medium is engaged to produce or release an aroma detected retro-nasally by the individual. The active medium may be engaged by saliva, components of saliva, or an initiating solution, as described above. The aroma may be detected retro-nasally, which can satiate the hunger of the individual. The aroma may cause other effects, which may depend on the type of active medium used the timing of its use, as described above.

The following Statements describe various embodiments of the present disclosure:

Statement 1. An oral appliance comprising: a body removably securable to one or more teeth: a chamber disposed on the body; and an active medium disposed in the chamber; wherein the active medium produces an aroma delivered retro-nasally.

Statement 2. The oral appliance of Statement 1, wherein the body is removably securable to one or more upper teeth.

Statement 3. The oral appliance of any one of the preceding Statements, wherein the body is removably securable to an anterior tooth and a posterior tooth.

Statement 4. The oral appliance of any one of the preceding Statements, wherein the body is removably securable to a left-side tooth and a right-side tooth on opposite sides of the mouth.

Statement 5. The oral appliance of any one of the preceding Statements, wherein the body includes a cross member which extends across the mouth from the left-side tooth to the right-side tooth.

Statement 6. The oral appliance of any one of the preceding Statements, wherein the one or more teeth are part of a denture or partial denture.

Statement 7. The oral appliance of any one of the preceding Statements, wherein the body includes a clasp which surrounds sides of the one or more teeth, such that occlusal surfaces of the one or more teeth are uncovered.

Statement 8. The oral appliance of any one of the preceding Statements, wherein the body is comprised of a biocompatible metal.

Statement 9. The oral appliance of any one of the preceding Statements, wherein the body comprises one or more of the following: a metal alloy, acrylic, polycarbonate, polyurethane, polyethylene, polyoxymethylene polyamide, nylon, stainless steel, vinyl, or composite.

Statement 10. The oral appliance of any one of the preceding Statements, wherein the body is manufactured based on a custom mold of the one or more teeth.

Statement 11. The oral appliance of any one of the preceding Statements, wherein the body is manufactured using molding, 3D laser sintering, 3D printing, stereolithography, or milling processes.

Statement 12. The oral appliance of any one of the preceding Statements, wherein the chamber comprises a biocompatible metal and/or a biocompatible plastic.

Statement 13. The oral appliance of any one of the preceding Statements, wherein the chamber is fixedly secured to the body.

Statement 14. The oral appliance of any one of Statements 1 to 12, wherein the chamber is removably secured to the body.

Statement 15. The oral appliance of any one of Statements 1 to 12, wherein the chamber is integrally formed with the body.

Statement 16. The oral appliance of any one of the preceding Statements, wherein the chamber is positioned parallel to the one or more teeth.

Statement 17. The oral appliance of any one of the preceding Statements, wherein the chamber has a rounded surface.

Statement 18. The oral appliance of any one of the preceding Statements, wherein the chamber comprises a sealable opening which, when open, is configured to receive the active medium, and when closed, is configured to retain the active medium within the chamber.

Statement 19. The oral appliance of any one of the preceding Statements, wherein the chamber comprises a porous surface, and the aroma produced by the active medium exits the chamber via the porous surface.

Statement 20. The oral appliance of any one of the preceding Statements, wherein the chamber comprises a first chamber and a second chamber separate from the first chamber.

Statement 21. The oral appliance of any one of the preceding Statements, wherein the first chamber and the second chamber are disposed on opposite sides of the mouth.

Statement 22. The oral appliance of any one of Statements 1 to 20, wherein the first chamber and the second chamber are disposed on the same side of the mouth.

Statement 23. The oral appliance of any one of the preceding Statements, wherein the active medium produces the aroma by interaction with an activator.

Statement 24. The oral appliance of any one of the preceding Statements, wherein the activator is saliva or a component of saliva.

Statement 25. The oral appliance of any one of Statements 1 to 23, wherein the activator is an initiating solution.

Statement 26. The oral appliance of any one of the preceding Statements, wherein the aroma is a hunger-satiating aroma.

Statement 27. The oral appliance of any one of the preceding Statements, wherein the active medium is a solid, gel, paste, or liquid.

Statement 28. The oral appliance of any one of the preceding Statements, wherein the active medium produces one or more of the following aromas: chocolate, vanilla, cinnamon, floral, butter, lemon, menthol, or mint.

Statement 29. A method of satiating hunger in an individual comprising: installing the oral appliance of any one of the preceding Statements in the mouth of the individual; and engaging the active medium to produce the aroma detected retro-nasally by the individual; wherein the aroma satiates the hunger of the individual when detected retro-nasally.

Although the present disclosure has been described with respect to one or more particular embodiments, it will be understood that other embodiments of the present disclosure may be made without departing from the scope of the present disclosure. Hence, the present disclosure is deemed limited only by the appended claims and the reasonable interpretation thereof.

What is claimed is:

1. An oral appliance comprising:
a body removably securable to one or more teeth;
a chamber disposed on the body; and
an active medium disposed in the chamber;
wherein the active medium produces an aroma delivered retro-nasally.

2. The oral appliance of claim 1, wherein the body is removably securable to one or more upper teeth comprising an anterior tooth and a posterior tooth.

3. The oral appliance of claim 1, wherein the body is removably securable to one or more upper teeth comprising a left-side tooth and a right-side tooth on opposite sides of the mouth, and the body includes a cross member which extends across the mouth from the left-side tooth to the right-side tooth.

4. The oral appliance of claim 1, wherein the one or more teeth are part of a denture or partial denture.

5. The oral appliance of claim 1, wherein the body includes a clasp which surrounds sides of the one or more teeth, such that occlusal surfaces of the one or more teeth are uncovered.

6. The oral appliance of claim 1, wherein the body is comprised of a biocompatible metal comprising one or more of the following: a metal alloy, acrylic, polycarbonate, polyurethane, polyethylene, polyoxymethylene polyamide, nylon, stainless steel, vinyl, or composite.

7. The oral appliance of claim 1, wherein the body is manufactured based on a custom mold of the one or more teeth using molding, 3D laser sintering, 3D printing, stereolithography, or milling processes.

8. The oral appliance of claim 1, wherein the chamber comprises a biocompatible metal and/or a biocompatible plastic.

9. The oral appliance of claim 1, wherein the chamber is fixedly secured to the body.

10. The oral appliance of claim 1, wherein the chamber is removably secured to the body.

11. The oral appliance of claim 1, wherein the chamber is integrally formed with the body.

12. The oral appliance of claim 1, wherein the chamber comprises a sealable opening which, when open, is configured to receive the active medium, and when closed, is configured to retain the active medium within the chamber.

13. The oral appliance of claim 1, wherein the chamber comprises a porous surface, and the aroma produced by the active medium exits the chamber via the porous surface.

14. The oral appliance of claim 1, wherein the chamber comprises a first chamber and a second chamber separate from the first chamber, and the first chamber and the second chamber are disposed on opposite sides of the mouth.

15. The oral appliance of claim 1, wherein the chamber comprises a first chamber and a second chamber separate from the first chamber, and the first chamber and the second chamber are disposed on the same side of the mouth.

16. The oral appliance of claim 1, wherein the active medium produces the aroma by interaction with saliva or a component of saliva.

17. The oral appliance of claim 1, wherein the active medium produces the aroma by interaction with an initiating solution.

18. The oral appliance of claim 1, wherein the active medium is a solid, gel, paste, or liquid.

19. The oral appliance of claim 1, wherein the active medium produces one or more of the following hunger-satiating aromas: chocolate, vanilla, cinnamon, floral, butter, lemon, menthol, or mint.

20. A method of satiating hunger in an individual comprising:
installing the oral appliance of any one of the preceding claims in the mouth of the individual; and
engaging the active medium to produce the aroma detected retro-nasally by the individual;
wherein the aroma satiates the hunger of the individual when detected retro-nasally.
